(19) United States
(12) Patent Application Publication
(10) Pub. No.: US 2026/0284425 A1
(43) Pub. Date: Sep. 24, 2026

(54) On-Eye Corneal Crosslinking for Control of Progressive Myopia and Treatment of Presbyopia

(71) Applicant: TECLens, Inc., Stamford, CT (US)

(72) Inventor: Patrick David Lopath, Stamford, CT (US)

(73) Assignee: TECLens, Inc., Stamford, CT (US)

(21) Appl. No.: 19/147,943

(22) PCT Filed: Jan. 12, 2024
(86) PCT No.: PCT/US2024/011429

§ 371 Date: Jul. 14, 2025

Related U.S. Application Data
(60) Provisional application No. 63/479,821, filed on Jan. 13, 2023.

(51) Int. Cl. A61N 5/06

(52) U.S. Cl. CPC A61N 5/062; A61N 2005/0626; A61N 2005/0632; A61N 2005/0648; A61N 2005/0661

(57) ABSTRACT

A method of treating progressive myopia or presbyopia in a patient includes performing crosslinking of at least some regions of a patient's cornea (1). The crosslinking is performed in an annular area (6) generally centered on the cornea and in a central area (7) inside the annular area. The crosslinking affects the refraction of the eye so as to change the focus on a peripheral region of the retina (3) by moving a portion of the focal surface (2) of the eye's image anteriorly (5) relative to the peripheral region, and the crosslinking affects the refraction in a central region (11) of the retina by moving a portion of the focal surface of the eye's image posteriorly direction relative to the central region. A device for performing the method includes a scleral contact lens including a UV emitter configured to emit UV light in the annular area and/or the central area.

### CROSS-REFERENCE TO RELATED APPLICATIONS

This application claims the benefit of the filing date of United States Provisional Patent Application No. 63/479,821, filed Jan. 13, 2023, the disclosure of which is hereby incorporated herein by reference.

### BACKGROUND OF THE INVENTION

Myopia has become an epidemic over the past 40 years. In 1970, 25% of the US population was nearsighted; today, that number is over 40%. Estimates place 5 billion people worldwide with myopia by 2050. Standard eyeglasses and contact lenses can correct most myopic refractive errors but do not impede the anatomical changes of progressive myopia typically seen in children such as axial elongation that can lead to further ophthalmic pathologies later in life. The American Academy of Ophthalmology's Task Force on Myopia has cited research that indicted these anatomical changes for increased risks of glaucoma, cataracts, retinal detachment, degenerative macular effects, myopic retinopathy, low vision, and blindness.

There is growing consensus that a lack of sufficient outdoor light at a young age is a primary trigger for the onset of progressive myopia. As the shift in global culture over the past few decades has significantly reduced young peoples' exposure to outdoor light, the prevalence of myopia has skyrocketed. Once initiated, pediatric progressive myopia is driven by a neurologic feedback loop sensitive to hyperopic image defocus at the periphery of the retina which triggers a physiologic compensation mechanism that cause the eye to grow in attempt to push the peripheral retina back to the focal surface of the image. However, because the eye grows as an oblate spheroid instead of sphere, this axial elongation only deepens the peripheral defocus, while pulling the central focus even farther in front of the fovea, making the patient more myopic. If not arrested, this process leads to an increased risk of pathologic myopia morbidities. Many approaches to this problem exist today, with most being daily (or nightly) wear lenses to create multifocal refractive adjustments (pulling the peripheral focus forward while pushing the central focus back). To be effective, these lens protocols require years of daily compliance.

Corneal crosslinking (CXL) with low intensity ultraviolet (UV) and riboflavin is the global standard of care for keratoconus (KC), typically stabilizing these pathologic corneas, and in most cases reducing the associated nearsightedness as the higher tensile strength crosslinked tissue causes the cornea to reshape. Many attempts have been made with traditional CXL systems to apply this reshaping effect to correct refractive error in healthy eyes. However, traditional CXL systems have no direct contact to the eye; they are “off-eye.” The “off-eye” approach prevents these CXL systems from being able to measure the crosslinking changes in real time, so these systems cannot monitor treatment progress or correct for patient-to-patient variability in response rate. Additionally, because these systems are not coupled to the patient, they are susceptible to mistargeting of the UV caused by patient motion. Both of these “off-eye” challenges prevent traditional CXL systems from delivering predictable refractive outcomes.

### BRIEF SUMMARY OF THE INVENTION

The inventions disclosed herein include methods and devices that leverage the corneal reshaping effect of CXL in a targeted and controlled manner to affect predictable refractive change aimed at slowing or stopping the progression of myopia, and, when necessary, improving the underlying residual myopia. These devices and methods can also be applied in older patients with presbyopia to improve reading distance vision. For the treatment of progressive myopia, the inventions desirably provide a permanent reduction in peripheral hyperopic defocus by turning the cornea itself into a multifocal lens using “on-eye” corneal crosslinking. For treatment of presbyopia, the inventions desirably provide some additional curvature to the center of the cornea to aid in the focusing on near objects. By being directly on the eye, the invention is believed to avoid the pitfalls of traditional CXL systems by enabling precise targeting and biomechanical feedback control of the crosslinking process. Using CXL to create a semi-permanent corneal shape similar to the temporary shapes created by other proven myopia progression control techniques desirably eliminates the protocol compliance and contact lens care challenges common with the pediatric patients most affected by progressive myopia. By adding additional curvature to a presbyopia patient's near vision eye, the invention can eliminate the patient's need for reading glasses.

The methods described herein desirably create a long-term change to the shape of the cornea in a manner that reduces or eliminates (depending on underlying severity) peripheral hyperopic defocus in a human eye for progressive myopia patients. A significant innovation is believed to be the use of corneal crosslinking (CXL) to affect peripheral refraction. Some previous attempts at refractive corneal shape change using corneal crosslinking (CXL), such as those for treatment of stable adult myopia, have targeted changes to the central refraction to change the patients' current visual acuity. The innovative system described herein preferably also achieves similar reduction or elimination of the underlying central myopia in a patient's eye (again, depending on the severity) to improve current visual acuity. However, a significant innovation is believed to be the use of CXL to control the shape of the cornea to change how the image is formed on the periphery of the retina to impact the evolution of the patient's vision over the long term. As described above, it is this peripheral retinal image that controls the axial growth mechanisms of the eye prior to ocular maturity.

According to one aspect of the present invention, a method of treating progressive myopia in a patient is provided. According to another aspect of the invention, a method of treating presbyopia in a patient is provided. Methods in accordance with aspects of the invention include performing crosslinking of at least some regions of the cornea of an eye of the patient.

According to some aspects of the invention, the crosslinking may be performed in at least one annular area of the eye. Preferably, that annular area is generally centered on the cornea. Also, according to some aspects of the invention, the crosslinking may be performed in a central area of the cornea. That central area may be inside the annular area.

Performing crosslinking according to aspects of the invention, particularly geared toward treatment of progressive myopia, desirably involves affecting the refraction of the eye in a manner so as to change the location of a focal surface with respect to peripheral regions of the retina. Such effect preferably moves a portion of the focal surface of the eye's image forwardly in an anterior direction relative to the peripheral region of the retina. According to some aspects of the invention, performing the crosslinking, particularly geared toward treatment of progressive myopia, desirably affects the refraction of the eye in a central region of the retina. Such affect to the refraction in the central area preferably moves a portion of the focal surface of the eye's image backwardly in a posterior direction relative to the central region of the retina.

According to any aspect of the present invention, the crosslinking may be UV-riboflavin mediated corneal crosslinking. Such crosslinking may include delivering ultraviolet light to the eye via a device comprising a scleral contact lens resting against the scleral surface of the eye. Moreover, in some aspects, the device may include a UV light emitter configured to emit the ultraviolet light in a predetermined pattern. Such predetermined pattern may comprise at least one annular area and/or a central area, where the central area may be inside the annular area. The method may further include independently adjusting the intensity of the ultraviolet light emitted in each of the central area and the annular area.

Other aspects of methods in accordance with the present invention may include creating a computational model based on the geometry and biomechanics of the eye. The computational model may be created based on the measurements of the three-dimensional shape of the cornea of the eye and at least one measurement of the biomechanical properties of the cornea. Yet other aspects of methods according to the invention may include using ultrasound to monitor changes in biomechanical properties of tissue in the eye during treatment.

Another aspect of the present invention provides a device for treating progressive myopia and/or a device for treating presbyopia. The device according to aspects of the invention includes an eye-contacting portion configured to rest against a scleral surface of an eye of the patient and includes a UV emitter supported by the contact portion and configured to emit ultraviolet light in a predetermined pattern. Depending on the refractive correction needs of the individual patient, such predetermined pattern desirably includes at least one annular area and/or a central area inside, where the central area may be inside the annular area.

According to some aspects of the invention, the UV emitter of the device may include a central diffuser configured to emit the ultraviolet light in the central area and at least one annular diffuser configured to emit the ultraviolet light in the respective annular area(s). Moreover, the amount of ultraviolet light emitted by each of the central diffuser and the annular diffuser(s) may be independently adjustable. Further, the device may include a plurality of optical fibers optically coupled to a central UV emitting region and to at least one annular UV emitting region, in order to supply the ultraviolet light to the central diffuser and the optical diffuser(s), respectively. According to some aspects of the invention, the device may include an optical mask to block some portion of the UV light from the source to create an annular pattern of UV light or an annular pattern in combination with a central area of UV light. Such patterns of UV light created via the optical mask may not be independently controllable.

The device in accordance with some aspect of the invention may include an ultrasound transducer configured to monitor biomechanical properties of tissue in the eye. According to some aspects, such ultrasound transducer may be arranged to monitor biomechanical properties of tissue irradiated by the ultraviolet light. According to yet other aspects, the ultrasound transducer may be arranged to monitor biomechanical properties of tissue irradiated by the ultraviolet light multiple separately irradiated areas of the cornea.

### BRIEF DESCRIPTION OF THE DRAWINGS

FIG. 1 is a cross-section of a patient's eye showing the focal surface before and after CXL treatment of progressive myopia according to an embodiment of the present invention.

FIG. 2 is a diagram of a basic crosslinking pattern, centered on the patient's visual axis, showing the geometric values that are iterated in a computational model for planning purposes.

FIG. 3 is a cross-sectional view of a scleral lens-based UV and ultrasound emitting crosslinking device according to an embodiment of the present invention, positioned on a patient's eye.

FIG. 4 is a perspective view of a scleral lens-based UV delivery device according to an embodiment of the present invention.

FIG. 5A is a top view of a map of UV intensity applied according to the present invention, as captured by a beam profiling camera.

FIG. 5B is a perspective view of the UV intensity map of FIG. 5A.

### DETAILED DESCRIPTION

### CXL Control of the Peripheral Focal Surface

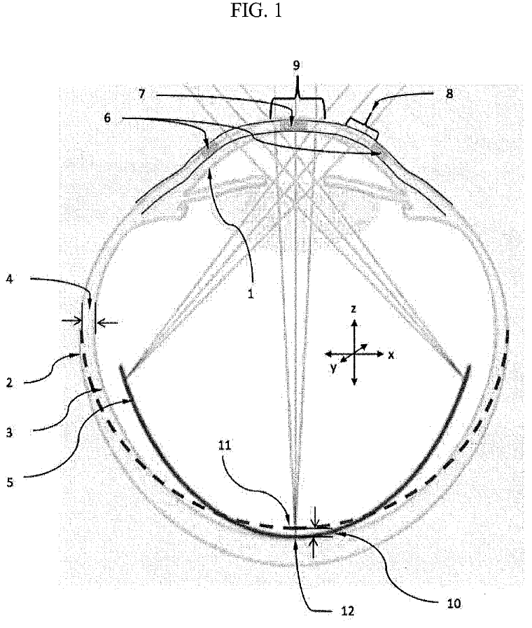

FIG. 1 shows a patient's eye with original corneal shape (1) that creates a focal surface (2) (shown with a dashed line) that is behind the retina (3) at the periphery by a distance (4).

This focus behind the retina is termed hyperopic defocus. This is the axial growth (z-direction) trigger that some aspects of the innovation aim to correct. The goal is to create a focal surface (5) (shown with a solid line) that is in front of the retina at the periphery. To change the peripheral retina focus, the mid-periphery of the cornea must be steepened. This may be done with corneal crosslinking by creating at least one annular pattern (6) of crosslinked stromal tissue at the periphery of the cornea along with a central spot (7) of crosslinked tissue. The stiffer crosslinked tissue better resists the intraocular pressure (IOP) versus the uncrosslinked mid-periphery. This causes the mid-periphery to bulge slightly forward, creating a more highly curved annulus of tissue (8) that shortens the focal length of light passing through it, and a flatter central region (9) that can reduce the current residual myopia by pushing the central focus back by some distance (10). The innovation desirably allows for individual titration of the UV dose between the annulus and the central spot, thus increasing control over the final corneal shape.

Without being limited to any particular theory of operation, crosslinking a central spot (7) of tissue in combination with the at least one annular region in the periphery of the cornea (6) creates a steep mid-peripheral region (8) that is significant in achieving a change in the focus in the periphery of the retina. Simply creating an annulus (6) of crosslinked tissue, without the central spot (7), the curvature of the cornea in a central region will increase, which increases myopia at the center of the retina (12). On the other hand, crosslinking only tissue in the central spot (7) reduces the curvature of the cornea in the central region (9), thus reducing residual myopia at the central retina (12). However, by crosslinking tissue in the central spot (7) in combination with an annular area (6), the mid-peripheral region (8) of the cornea between the central spot (7) and the annular pattern (6) becomes more highly curved, affecting the refraction in the periphery of the retina (3), specifically by increasing myopia at the periphery of the retina (3). Moreover, proper selection of the sizes of the annular and central crosslinked regions and proper titration of the amount of crosslinking in each of the central spot (7) and the annular pattern (6) allows control over how much peripheral myopia is created and how much the residual central myopia is reduced.

### Computational Modeling

One of the significant procedures in the innovative method to control progressive myopia and treat presbyopia with crosslinking is the use of computational pre-planning. That is, to optimize the outcome for each patient, measurements of the patient's cornea are made prior to treatment and used as inputs into a computational plan. The three-dimensional corneal shape (e.g., corneal tomography) and a scalar estimate of the tensile modulus (e.g., using Goldmann Tonometry or other type of biomechanical assessment tool) are collected as inputs to a finite element model of each patient's eye—δin essence, creating a digital twin of the eye to be treated. This model may then be used to calculate the post-procedure corneal refractive shapes that result from application of a wide range of therapeutic crosslinking parameters.

The computational model calculates the possible refractive outcomes by changing the biomechanical parameters as a function of the simulated UV dose in various patterns defined by the control radii of the peripheral CXL annulus (R1, R2) and the central spot diameter (Dia). Changes to the biomechanics change the refractive shape because, as the corneal biomechanics change, the equilibrium between the tissue strength and the intraocular pressure that defines the shape of the cornea also changes. The treatment parameters (values of R1, R2, Dia, and the independently-controlled post-crosslinking tensile moduli in the two irradiated regions) may be tuned to optimize the refractive shape by iterating through the range of available treatment parameters in the finite element simulation and assessing the effect of each set of parameters on the digital twin prior to treating the patient. In some embodiments of the invention, the refractive effect (the focal surface) of the simulated corneal shape may be confirmed with computational ray tracing. Preferably, one output of the computational model is a control value that can be measured during the crosslinking therapy and that is linked to the optimum final refractive shape. In the preferred embodiment, this control value is the percentage change in the scalar tensile modulus that can be assessed indirectly with the perioperative ultrasound system in this invention.

The computational model is not able to simulate how each particular patient's tensile modulus will change as a result of applied UV dose, as that response rate varies from patient to patient. To compensate for this patient-to-patient variability in crosslinking response, the changing tensile modulus is monitored in real time and compared to the control value from the simulation.

### The Device

To create the annular and central UV patterns, any UV emitter in the range of 360 nm to 380 nm can be used, with a mask in the form of a contact lens with UV opaque regions to define the pattern. The opaque regions can be variably opaque to facilitate titration of UV dose between the annulus and the central spot.

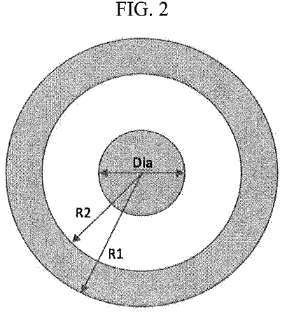

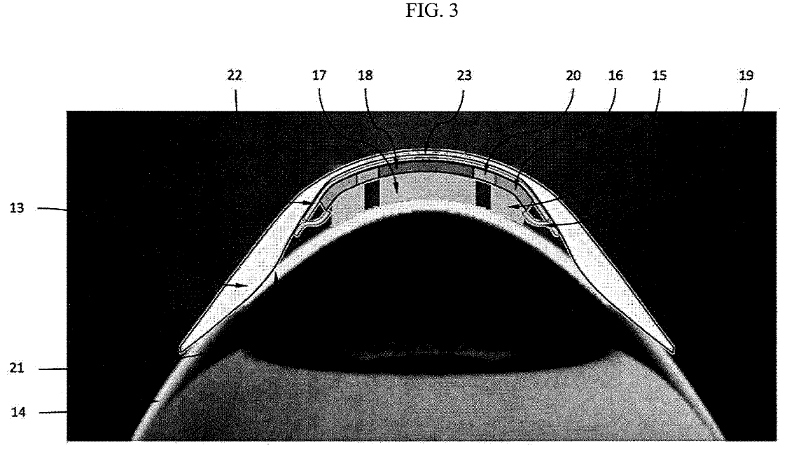

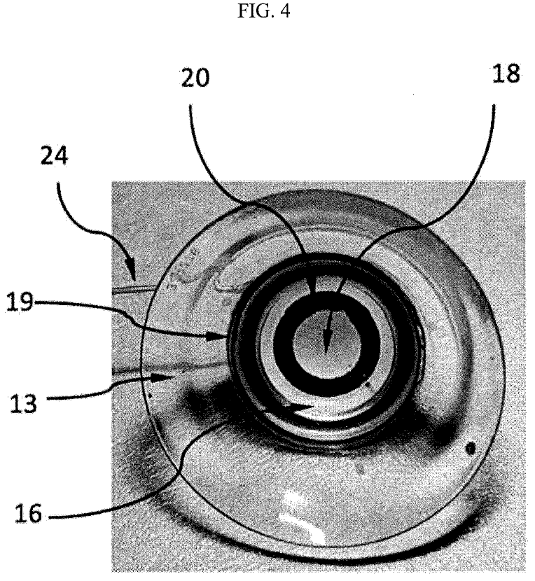

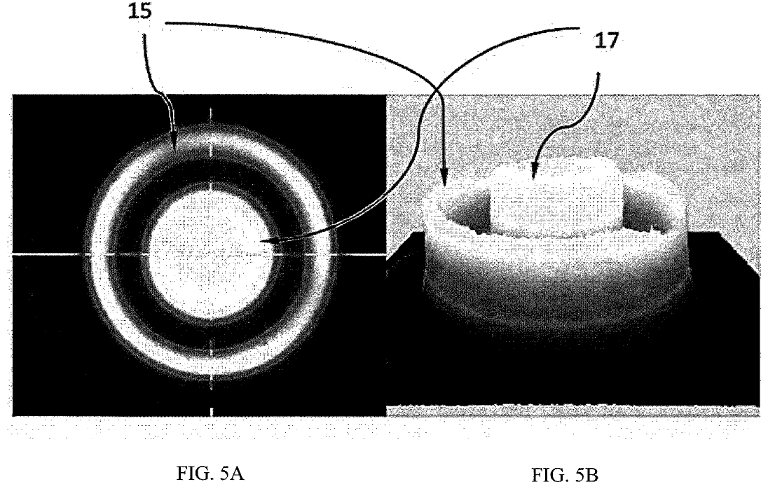

Ideally, a device as shown in FIGS. 3 and 4, including a scleral mating surface (13) configured to sit on the eye of a patient (14), is used to deliver and pattern the UV energy. The scleral interface desirably ensures that the UV pattern stays aligned concentric with the patient's visual axis. The device is configured to emit UV light in a controlled manner in at least 1 of multiple regions: one or more annuli around the visual axis (15) from an annular UV emission region (16) and a central spot (17) from a central UV emission region (18). Such UV emission regions may include optical diffusers. Typically, UV doses range from 1.0 J/cm² to 10.0 J/cm² . The annular pattern is emitted with its outer edge defined by the aperture reflector (19) having an inner radius R1 and its inner edge defined by the annular gap (20) having an outer radius R2 (see FIG. 2). The central spot is emitted with its outer edge defined by the inner diameter of the annular gap (i.e., Dia from FIG. 2). The light is directed down onto the patient's riboflavin saturated cornea (21) by the scattering of the optical diffusers (16, 18) and reflection from the top reflector (22). Titration of the UV dose levels delivered to annulus and the central spot may be managed by a control system with the UV energy delivered to the lens by optical fibers (24).

The annular gaps that define the annulus and central spot UV patterns may be created in different manners in different embodiments of the device. In some embodiments it may be desirable to have independent control over the intensity and duration of the light emitted from the annulus and the central spot. In these cases, the two regions may be connected to independent UV source channels. In other embodiments, a single UV source channel can be used, and the annular gaps can be defined by a simple pattern mask that blocks the light that would otherwise irradiate the cornea in areas outside the defined annulus and/or spot. In these embodiments, the underlying light emitter can be standardized, with the pattern mask as an interchangeable ‘snap in’ component, allowing the physician to choose a UV pattern that aligns with an optimized treatment plan.

As the UV crosslinks the cornea, the changing corneal biomechanical properties of the tissue are monitored using an ultrasound transducer (23) over the central spot, and, in some embodiments, over the peripheral annuli or in both regions.

### The Method for Progressive Myopia

Progressive myopia is typically diagnosed at a young age (i.e., pre-teen), when the patient presents with nearsightedness. At initial screening for potential treatment, the central and peripheral refractive characteristics of progressive myopia are confirmed with standard measurement techniques (e.g., retinoscopy, autorefraction, etc.) and a baseline axial length is taken with ultrasound and/or OCT. The patient may be fitted with a contact lens or spectacles to assess the necessary refractive change that will later be applied directly to the cornea via CXL. This will allow a pre-test and a set of measurements to confirm the required central and peripheral refraction changes.

Once the decision for CXL intervention is made, the patient's corneal tomography and IOP are measured prior to intervention. Goldmann tonometry (or another biomechanical evaluation, as per physician preference) is performed, typically with the Orssengo-Pye adjustments made (or similarly appropriate adjustments for alternate biomechanical evaluation techniques), to yield a scalar tensile modulus baseline value. These data are uploaded to a computational model that maps the patient's modulus and IOP onto a finite element mesh of the patient's tomographic data. The doctor also inputs the patient's current manifest refraction and desired refractive outcome. The model then iterates through the possible CXL treatments, applying them to the digital eye twin, calculating the output refractive corneal shapes, and ray tracing the focal surfaces. A selection of the optimal results are provided to the physician as a set of treatment control paraments, including the necessary design of the particular UV delivery patterned scleral lens to select.

The patient returns to the physician on treatment day. The doctor delivers the necessary riboflavin to the eye then applies the selected treatment scleral UV delivery lens and inputs the control parameters into the treatment system. The device is then used to deliver and pattern the UV energy, as discussed above, as well as to monitor the changing corneal biomechanical properties as the UV energy crosslinks the cornea.

Once the treatment is complete, the patient is provided analgesic drops, a pair of UV blocking sunglasses, and goes home. The refractive outcome is checked at approximately 1 month, 3 months, and 6 months. Over the following years, at the physician's discretion based on the patient's history of progression, the patient's corneal shape is checked with tomography and refraction is checked with autorefraction or retinoscopy or other suitable technique. The axial length of the eye is again measured. If the patient continues to progress myopically, the treatment can be repeated if desired.

### The Method for Presbyopia

The procedure method and patient flow from initial screening through treatment for presbyopia is the same as the treatment for progressive myopia. However, the type of correction applied may be different. Today, many patients who receive IOLs (intraocular lenses) as treatment for cataracts are given what is termed ‘mini-monovision’ with the IOL selected so that the patient's eyes are about 1.0 D to 1.5 D apart in manifest refraction. Typically, the patient's non-dominant eye is selected to have slightly more refractive power for better near vision. This same technique is one approach used in the CXL correction of presbyopia. However, instead of choosing a higher power IOL for the non-dominant eye, the cornea in the non-dominant eye is corrected to be more highly curved (by 1.0 D to 1.5 D) than the dominant (distance vision) eye. In this manner, the patient is able to see both up close and at distance, with the brain automatically adjusting to use the image from the in focus eye.

There are other approaches to presbyopia correct in use in the clinic today. One common practice is to treat presbyopia with multifocal contact lenses that are designed to provide clear vision at distance vision through the center of the lens, and clear reading vision through the mid-periphery of the lens. These are termed ‘center-distance’ lenses. The CXL approach presented above to reshape the cornea for the treatment of progressive myopia with both annular and central UV crosslinked regions effectively creates a center-distance shape on the cornea. Thus, the invention presented herein is effective at treating presbyopia, or patients that are myopic before treatment.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of treating progressive myopia in a patient, comprising:
performing crosslinking of at least some regions of the cornea of an eye of the patient.

2. (canceled)

3. The method of claim 1, wherein the crosslinking is performed in at least one annular area of the eye.

4. The method of claim 1, or wherein the crosslinking is performed in at least one annular area generally centered on the cornea.

5. The method of claim 1, wherein the crosslinking is performed in a central area of the cornea inside the at least one annular area.

6. The method of claim 1, wherein the crosslinking is performed in a central area of the cornea.

7. The method of claim 1, wherein the performing of the crosslinking affects the refraction of the eye in a manner that changes the focus on a peripheral region of the retina.

8. The method of claim 7, wherein the refraction of the eye is affected by moving a portion of the focal surface of the eye's image forwardly in an anterior direction relative to the peripheral region of the retina.

9. The method of claim 1, wherein the performing of the crosslinking affects the refraction of the eye in a central region of the retina.

10. The method of claim 9, wherein the refraction of the eye is affected by moving a portion of the focal surface of the eye's image backwardly in a posterior direction relative to the central region of the retina.

11. The method of claim 1, wherein the crosslinking is UV-riboflavin mediated corneal crosslinking.

12. The method of claim 11, wherein the step of performing crosslinking includes delivering ultraviolet light to the eye via a device comprising a scleral contact lens resting against the scleral surface of the eye.

13. The method of claim 12, wherein the device includes a UV light emitter configured to emit the ultraviolet light in a predetermined pattern, the predetermined pattern comprising at least one annular area

14. The method of claim 12, wherein the device includes a UV light emitter configured to emit the ultraviolet light in a predetermined pattern, the predetermined pattern comprising a central area.

15. The method of claim 12, wherein the device includes a UV light emitter configured to emit the ultraviolet light in a predetermined pattern, the predetermined pattern comprising at least one annular area and a central area inside the at least one annular area.

16. The method of claim 15, further comprising independently adjusting the intensity of the ultraviolet light emitted in each of the central area and the at least one annular area.

17. The method of claim 1, further comprising creating a computational model based on the geometry and biomechanics of the eye.

18. The method of claim 17, wherein the computational model is created based on measurements of the three-dimensional shape of the cornea of the eye and at least one measurement of the biomechanical properties of the cornea.

19. The method of claim 1, further comprising using ultrasound to monitor changes in biomechanical properties of tissue in the eye.

20-27. (canceled)

28. The method of claim 5, wherein an inner radius of the at least one annular area is radially spaced outwardly away from an outer radius of the central area.

29. The method of claim 1, wherein the performing of the crosslinking affects the refraction of the eye by moving a first portion of the focal surface of the eye's image forwardly in an anterior direction in a peripheral region of the retina while moving a second portion of the focal surface of the eye's image backwardly in a posterior direction in a central region of the retina.
